(19) United States
(12) Patent Application Publication
(10) Pub. No.: US 2021/0186215 A1
(43) Pub. Date: Jun. 24, 2021

(54) STABILITY AND ANTI TIPPING DEVICE

(71) Applicant: PurpleSun Inc., Long Island City, NY (US)

(72) Inventor: Luis F. Romo, New York, NY (US)

(21) Appl. No.: 16/769,919

(22) PCT Filed: Dec. 4, 2018
(86) PCT No.: PCT/US18/63769

§ 371 Date: Jun. 4, 2020

Related U.S. Application Data
(60) Provisional application No. 62/594,440, filed on Dec. 4, 2017.

(51) Int. Cl. A47B 97/00

(52) U.S. Cl. CPC A47B 97/00; A47B 2097/008

(57) ABSTRACT

Stability devices of varied shape and configurations that serve the function of preventing the systems and equipment such as disinfection equipment or other types of structures and systems from tipping over due to lateral loading. These stability devices attach to the base of the disinfection device and sit slightly above floor level such that mobility of the device is unhindered when in motion but that keep the device from toppling past a certain maximum angle from the vertical when lateral loads are applied at the top of the disinfection device. The stability devices provide protection to the operators and to the equipment and thereby facilitate the function of such disinfection devices in hospitals and other facilities.

### CLAIM OF PRIORITY

This patent application claims the benefit of priority, under 35 U.S.C. Section 119(e), to Luis Romo U.S. Patent Application Ser. No. 62/594,440, entitled “STABILITY AND ANTI TIPPING DEVICES,” filed on Dec. 4^{th}, 2017 (Attorney Docket No. 5150.004PRV), each of which is hereby incorporated by reference herein in its entirety.

### BACKGROUND

Microbial contamination is a global concern within many industries, especially in the healthcare industry. It costs countries billions of dollars in expenses per year, and, more importantly, the contaminant pathogens plague private and public (e.g., healthcare) settings and surroundings. These contaminated surroundings lead to infections and may ultimately cause deaths.

The current time-tested technology of cleaning surfaces with chemical disinfectants has held infections in check for over a century but the almost intractable problem of surgical site infections has spawned numerous attempts to develop newer, improved methods of cleaning hospital surfaces in order to reduce the number of infections and save costs. Prominent among the technologies that have been developed in an attempt to reduce the rate of hospital-acquired infections (HAIs) is ultraviolet (UV) light, which has been proven effective in laboratories but for which implementation in hospitals has been hindered by a lack of practical design implementations and concerns about UV hazards, Indeed, existing area disinfection devices can be tall and awkward to move, and may tip over during transfer to and from the application of too high a lateral load. Therefore, it is desirable to provide techniques and devices that facilitate ease of transportation and stability of disinfection devices during deployment, and to avoid creating any tipping hazards.

Other patents and publications which may be related to disinfection devices include: U.S. Pat. Nos. 8,907,304; 9,044,521; TW381489Y; TW556556Y; U.S. Pat. No. 7,459,964; CN206063449; WO2010115183; US20150284266; KR101767055; WO2015012592; and KR20150028153.

### SUMMARY OF THE INVENTION

The present invention generally relates to a stability device for the support in operation, transportation and stability of mechano-electrical devices and cabinets and tall paneling systems or lighting systems. An exemplary embodiment of a disinfection system is disclosed in U.S. Pat. No. 9,675,720 incorporated by reference.

In some embodiments, the application describes means and methods for assuring that the present invention of the stability device be installed or affixed to and for structures, equipment, devices or systems together defined as systems is desired because of unintentional tip over of systems during transfer from the application of lateral loads during operation or transport, therefore it is desirable to provide techniques that facilitate ease of transportation and stability during operations, and to prevent any tipping hazards during operation for example in a healthcare environment. The stability device allows for operation of the system in numerous geometric configurations. An example of such a system can be at least a disinfection device as referenced in prior art U.S. Pat. No. 9,675,720, previously incorporated by reference. For the purpose of clarity and description for this invention the systems will be referred to as a disinfection system. In some embodiments, a central column is the main support structure for the disinfection systems and the column stability device represents one of three or more stability devices that provide stability to the disinfection systems and assure that the entire system can withstand a lateral load without tipping over of the system.

Exemplary embodiments may address one or more of the problems and deficiencies of the art discussed above. However, exemplary embodiments may additionally or alternatively prove useful in addressing other problems and deficiencies in several technical areas. Therefore, the scope of embodiments should not necessarily be construed as being limited to addressing any of the particular problems or deficiencies discussed herein.

Some embodiments of the presently-disclosed disinfection system and methods have several features, no single one of which is solely responsible for their desirable attributes.

In accordance with exemplary embodiments, the stability device is a component of variable shapes, one or more of which attach around the base of disinfection systems to thereby prevent the systems from tipping over as the result of laterally applied forces. Exemplary embodiments of the stability device include a stability device, a wing stability device, and a column lateral stability device which vary in shape due to their location and the potential lateral or vertical forces to which the stability devices may be subject.

In accordance with the exemplary embodiments, the deployed stability device stabilizers will be of such a design so as to avoid or mitigate the creation of any tripping hazards or otherwise cause interference to any operators or personnel. In some embodiments, this result is made effective by ensuring that no part of the stability devices extend radially outward and past the body or main structure of the disinfection systems.

In accordance with the exemplary embodiments, the stability device has a well-defined function but can have different shapes and dimensions depending on which part or location of a disinfection system the stability devices are a permanently or temporarily affixed. In some embodiments, there are three basic shapes for the stability devices (stability device, wing stability device, and column lateral stability device) that have unique dimensions and these are as described hereafter in the figures, Each type of stability device serves the same basic purpose of inter alia, preventing tip-over of systems, but is uniquely crafted to suit the particular location on the disinfection systems where attached.

The following, non-limiting examples, detail certain aspects of the present subject matter to solve the challenges and provide the benefits discussed herein, among others.

Example 1 is a stability device connectable to a disinfection device, the stability device comprising: a plate including a plurality of bores, the plate configured to connect the stability device with a base of a disinfection device using the plurality of bores; a bottom flange substantially parallel with the plate and spaced away therefrom; a first center flange, a second center flange, and a third center flange each of the first center flange, the second center flange, and the third center flange connected to the plate and the bottom flange; and a bumper connected to a bottom portion of the bottom flange and configured to contact a floor when the disinfection device tips.

In Example 2, the subject matter of Example 1 optionally includes a stability device cover connected to the plate, the bottom flange, the first center flange, the second center flange, and the third center flange.

In Example 3, the subject matter of any one or more of Examples 1-2 optionally includes wherein the first center flange, the second center flange, and the third center flange extend between the bottom flange and the plate at an angle of about 45 degrees.

In Example 4, the subject matter of any one or more of Examples 1-3 optionally include wherein first center flange, the second center flange, and the third center flange each have a geometric shape of a trapezoid.

Example 5 is a stability device connectable to a disinfection device, the stability device comprising: a plate configured to connect the stability device with a base of a disinfection device; a first lateral flange substantially parallel with the plate and spaced away therefrom; a second lateral flange substantially parallel with the plate and spaced away therefrom; and a first center flange, a second center flange, and a third center flange each of the first center flange, the second center flange, and the third center flange connected to the plate, the first lateral flange and the second lateral flange.

In Example 6, the subject matter of Example 5 optionally includes a first bumper connected to a bottom of the first lateral flange and configured to contact a floor when the disinfection device tips laterally in a first direction.

In Example 7, the subject matter of any one or more of Examples 5-6 optionally include a second bumper connected to a bottom of the second lateral flange and configured to contact a floor when the disinfection device tips laterally in a second direction.

In Example 8, the subject matter of any one or more of Examples 5-7 optionally include wherein the first lateral flange is substantially coplanar with the second lateral flange.

Example 9 is a stability assembly connectable to a disinfection device, the stability assembly comprising: a column base including a top portion and a bottom portion opposite the top portion; a column framework extending substantially vertically from the top portion of the column base; a stability device connected to the bottom portion of the column base; a lateral stability device connected to the bottom portion of the column base and spaced away from the stability device.

In Example 10, the subject matter of Example 9 optionally includes wherein the stability device is positioned at a front portion of the bottom portion of the column base and wherein the lateral stability device is positioned at back portion of the bottom portion of the column base.

In Example 11, the subject matter of any one or more of Examples 9-10 optionally include wherein the lateral stability device further comprises: a lateral plate configured to connect the lateral stability device to the column base; a first lateral flange substantially parallel with the lateral plate and spaced away therefrom; a second lateral flange substantially parallel with the lateral plate and spaced away therefrom; and a first center flange, a second center flange, and a third center flange each of the first center flange, the second center flange, and the third center flange connected to the plate, the first lateral flange and the second lateral flange.

In Example 12, the subject matter of Example 11 optionally includes wherein the stability device further comprises: a stability plate configured to connect the stability device to the column base; a bottom flange substantially parallel with the stability plate and spaced away therefrom; a first stability center flange, a second stability center flange, and a third stability center flange each of the stability first center flange, the stability second center flange, and the stability third center flange connected to the stability plate and the bottom flange; and a bumper connected to a bottom portion of the bottom flange and configured to contact a floor when the stability assembly tips.

Example 13 is a method of assembly a stability device connectable to a disinfection device, the method comprising: providing a plate including a plurality of bores, the plate configured to connect the stability device with a base of a disinfection device using the plurality of bores; providing a bottom flange configured to be assembled in an arrangement substantially parallel with the plate and configured to be spaced away therefrom; connecting a first center flange to the plate and the bottom flange; and connecting a bumper to a bottom portion of the bottom flange, the bumper configured to contact a floor when the disinfection device tips.

In Example 14, the subject matter of Example 13 optionally includes connecting a second center flange to the plate and the bottom flange.

In Example 15, the subject matter of Example 14 optionally includes connecting a third center flange to the plate and the bottom flange.

In example 16, the apparatuses or method of any one or any combination of Examples 1-15 can optionally be configured such that all elements or options recited are available to use or select from.

Each of the following elements can be combined with any of the previous discussed examples.
1. Stability devices that can be affixed to a system to prevent tipping of the system from lateral or vertical forces in numerous configurations of the system.
2. Stability devices that provide anti tipping capabilities in numerous configurations of the systems of which the devices are permanently attached.
3. Stability devices that provide anti tipping capabilities of which they are temporarily attached to systems.
4. Stability devices that are comprised of a material which are not casters.
5. Stability devices that are utilized to counter-act numerous lateral or vertical forces being applied on the system in a variety of directions.
6. Stability devices that can be automated with a motor.
7. Stability devices that can be elevated with actuators or motorized gears in response to counter directional forces to prevent tipping.
8. A set of any number of stability devices that, attached at the base of a disinfection system or any other structure, collectively prevent the structure from tipping over too far under lateral loading imposed on the system in numerous configurations of the stability device and the disinfection system.
9. Stability devices attached at the base of a disinfection system, being portable in a compact configuration and being protected from tipping too far by the presence of stability devices that are attached to the base of said disinfection system a small distance above the floor designed to prevent lateral loads from tipping the device and causing undue hazards to operators or workers.
10. Stability devices that have electro mechanical sensors and indicators to alert the user upon activation.
11. A method of preventing, by design, a tipping hazard due to a relatively large, tall and portable system or other portable or non-portable structures.
12. In various embodiments, devices and methods of the present invention form a part of a portable disinfection systems configured to use ultraviolet light to disinfect rooms, areas, surfaces, and equipment in hospital and other facilities.
13. A method about where lateral and vertical forces are applied from the left direction to the system and the column stability devices and lateral stability devices are activated to counteract the forces to prevent tipping.
14. A method about where lateral and vertical forces are applied from the right direction to the system and the column stability devices and lateral stability devices are activated to counteract the forces to prevent tipping.
15. A method about where lateral and vertical forces are applied from the front direction to the system and the column stability devices and lateral stability devices are activated to counteract the forces to prevent tipping.
16. A method about where lateral and vertical forces are applied from the back direction to the system and the column stability devices and lateral stability devices are activated to counteract the forces to prevent tipping.
17. The stability device can be permanently positioned to make contact with the floor.
18. The stability device can hover over the floor to provide mobility to the system and only be utilized or activated during a tip event or through control from a controller.

These and other embodiments are described in further detail in the following description related to the appended drawing figures.

### BRIEF DESCRIPTION OF THE DRAWINGS

The novel features of the invention are set forth with particularity in the appended claims, A better understanding of the features and advantages of the present invention will be obtained by reference to the following detailed description that sets forth illustrative embodiments, in which the principles of the invention are utilized, and the accompanying drawings of which:

FIG. 1 shows a perspective view of an exemplary embodiment of a stability device assembly.

FIGS. 2A-2D show multiple perspectives of an exemplary embodiment of a stability device assembly.

FIG. 3 shows a perspective view of an exemplary embodiment of a wing stability device assembly.

FIGS. 4A-4D show multiple perspectives of a wing stability device assembly.

FIG. 5 shows a perspective view of an exemplary embodiment of a column lateral stability device assembly.

FIGS. 6A-6D shows multiple perspectives of an exemplary embodiment of a column lateral stability device assembly.

FIG. 7 shows a section of an exemplary embodiment of a column lateral stability device.

FIG. 8 shows an end view of an exemplary embodiment of a stability device on an exemplary embodiment of a disinfection device.

FIG. 9 shows a side view of an exemplary embodiment of a panel with exemplary embodiments of both a stability device and the lateral stability device.

FIG. 10 shows an overhead wireframe view of the base of an exemplary embodiment of a stability device.

FIG. 11 shows a side view of an exemplary embodiment of a panel with an exemplary embodiment of a wing stability device.

FIG. 12 shows a side view of the base of an exemplary embodiment of a column with exemplary embodiments of both a stability device and a lateral stability device.

FIG. 13 shows a schematic side view of the column and the wing stability device, with basic dimensions indicated and a figurative lateral force applied to the top of the column.

FIG. 14 shows a schematic side view of the column leaning at the maximum tipping angle from a lateral force applied at the top.

### DETAILED DESCRIPTION OF THE INVENTION

Specific embodiments of the disclosed device, delivery system, and method will now be described with reference to the drawings. Nothing in this detailed description is intended to imply that any particular component, feature, or step is essential to the invention.

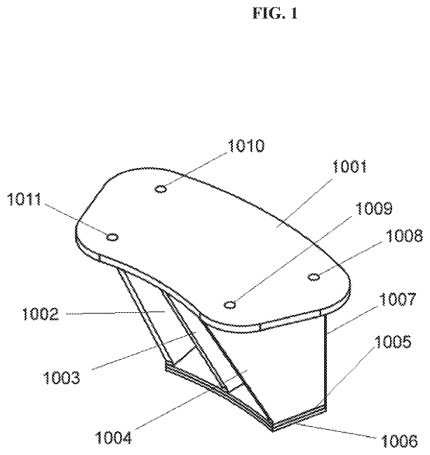

FIG. 1 shows a perspective view of an exemplary embodiment of a stability device. The stability device comprises a stability device top flange 1001, three stability device center flanges 1002, 1003, and 1004, a stability device bottom flange 1005, a stability device bumper 1006, and a stability device cover 1007. The top flange 1001 is a flat planar plate that is disposed on top of the stability device assembly with a surface or edges that extend past the rest of the assembly and which has four holes 1008, 1009, 1010, and 1011 disposed near the four corners of the plate. The holes are configured to couple the stability device with the base of a disinfection device. When attached to the base of a disinfection device, the stability device helps prevent the device from tipping over under normally applied lateral loads at the top of the device. The corners of the top flange 1001 are rounded to avoid sharp edges from presenting hazards. Optionally, in this and other embodiments, there can be at least two holes in the stability device top flange and as many as might be necessary to provide a firm attachment. Optionally, in this and other embodiments, the plate may be any shape that provides for a firm attachment and fits in the allotted space, including but not limited to rectangles, triangles, circles, or irregular shapes. Optionally, in this and other embodiments, the holes may be located in any set of two or more locations that provide a firm attachment to the base of the disinfection unit. Optionally, in this and other embodiments, the holes can be located at the corners of the base plate, the sides of the base plate, or any other location around the perimeter of the base plate or in the center such that a firm attachment is provided for Optionally, in this and other embodiments, any fixation element can be used to couple the holes of a column assembly with a disinfection device including inter alfa, screws, rivets, bolts, weldments, etc. Optionally, in this and other embodiments, the stability device components are composed of either aluminum or polypropylene or other comparable material with a black powder or comparable coat or any combination therein. Optionally, in this or other embodiments, the material of construction may be UV-proof or coated with UV-absorbing materials.

One end of each of the three center flanges 1002, 1003, and 1004 are coupled to the bottom surface of the top flange 1001. The attachment of the stability devices to the bottom surface of the column or panels may be accomplished by any one of a number of commonly known attachment methods including, but not limited to nuts and bolts, screws, weldments, glue, electromagnetic coupling, mechanical coupling, or any comparable mechanism. The stability device may, in this and other embodiments, fit between the vertical space beneath the base of the disinfection device with enough clearance above the floor to allow free movement of the disinfection device. Optionally, in this and other embodiments, the total height of the stability device may be on the order of 2-3 inches or more while the width may be twice this dimension or more, but neither the height nor the width is limited to these dimensions. Optionally, in this and other embodiments, the distance between the bottom of the stability device and the floor may be on the order of approximately 1/8 inch. The center flanges are trapezoidal in one embodiment but are not limited to this shape and may be rectangular, square, or any other shape that provides structural integrity. The center flanges are flat plates in one embodiment but may have other cross-sections such as cylindrical, tubular, channels, or any other cross-section that provides structural integrity. Optionally, in other embodiments, there may be one or more center flanges,

The other end of the center flanges is coupled to the top surface of a flat bottom flange 1005 which is a slightly curved rectangle in the current embodiment but may be almost any arbitrary shape that suits the purpose of providing a rigid base to hold the three center flanges together. The bottom flange 1005 has a smaller profile than the top flange in this embodiment to minimize any obstruction it may present at the floor level, and because it need only contact the floor during a tipping event in order to stop the disinfection device from tipping, but it could have almost any arbitrary size and shape that allowed it to perform the same function. The bottom flange, and the attached stability device bumper which has the same dimensions, have a narrower profile at the bottom to prevent it from protruding outwards and posing a tripping hazard, but these flanges could, in other embodiments, protrude from under the base of the column to provide added stability at the cost of a minor tripping hazard.

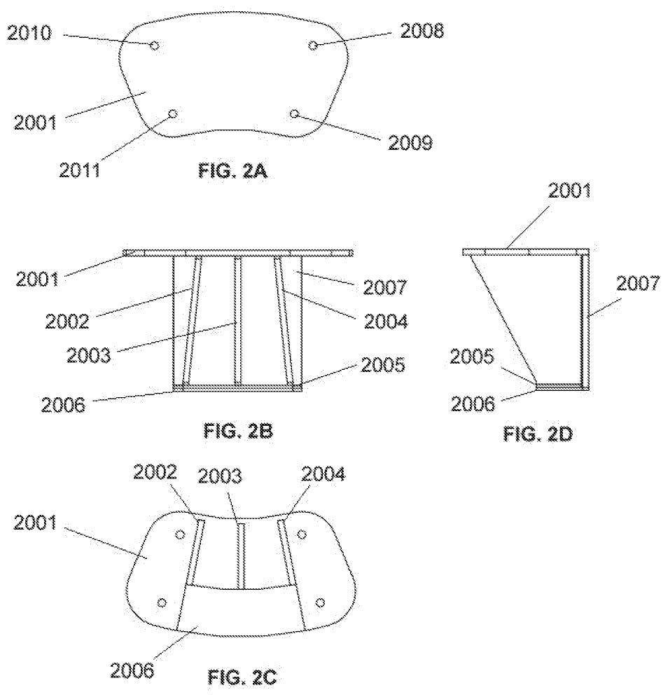

FIGS. 2A through 2D show multiple perspectives of an exemplary embodiment of a stability device assembly. FIG. 2A illustrates a top view of the assembly in which only the top flange 2001 is visible along with the four attachment screw holes 2008, 2009, 2010, and 2011. The top flange has rounded edges and conforms to the curvature of the base plate to which it is attached. Optionally, in this and other embodiments, the top flange make have alternate shapes as long as the shape provides a structurally rigid connection between the stability device and the base plate of the column to which it is attached. The number of components to the stability device, 7 in the instant embodiment, does not represent a minimum number of parts but merely represents a convenience from the point of view of the assembly of the stability device, and it is possible that the entire stability device could be manufactured from a single piece of metal, plastic, or other material. FIG. 2B illustrates an inside view or back view of the stability device showing the top flange 2001, the bottom flange 2005, the bottom bumper 2006, the three center flanges 2002, 2003, and 2004, and the stability device cover 2007 which is visible from the exterior. In this current embodiment, none of the parts is optional as they all serve to add rigidity and strength to the basic structure, although alternate manufacturing techniques may enable one or two of these parts to be forgone in lieu of other parts being substituted that have increased girth or material strength. The bottom bumper, for example, could be omitted if the bottom flange was made thicker or stronger, and the middle center flange could be omitted if the other flanges and their attachments to the flanges were sufficiently strengthened to take up the design load. FIG. 2C illustrates a bottom view of the stability device showing the primary visible components, the bottom bumper 2006, the three center flanges 2002, 2003, and 2004, and the top flange 2001. It can be seen in FIG. 2C that the placement of the four holes (2008, 2009, 2010, and 2011 in FIG. 2A) is limited by the location of the three center flanges which necessitate the screw holes being outwards towards the edges although this in no way eliminates the possibility of the screw holes being located elsewhere. Optionally, in this and other embodiments, the stability device bumper on the bottom provides the primary point of contact between the stability device and the floor in the event the column should tip. Optionally, in this and other embodiments, the stability device bumper will assume the weight of the load placed upon it during any tipping event and will distribute the weight through the connecting flanges and the stability device cover. FIG. 2D illustrates a side view of the stability device bumper showing the top flange 2001, the stability device cover 2007, the bottom flange 2005, and the stability device bumper. This image illustrates how, in this and other embodiments, the profile of the stability device narrows towards the floor. Optionally, in this and other embodiments, the forces transmitted from the floor to the surface area of the stability device bumper will diminish at the column base in inverse proportion to the surface area of the top flange. Optionally, in this and other embodiments, the entire stability device assembly absorbs the force of the weight placed upon it by the column and delivers this force to the floor.

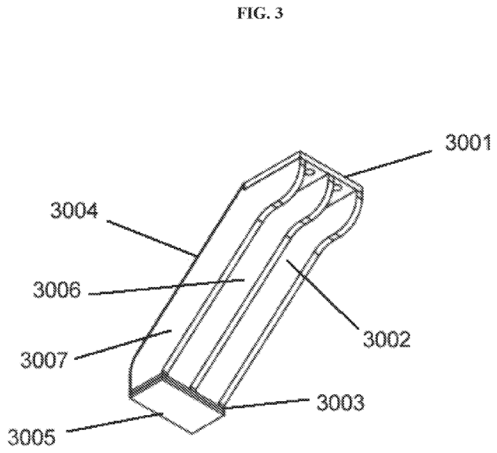

FIG. 3 shows a perspective view of an exemplary embodiment of a wing stability device which is comprised of a wing stability device top flange 3001, three wing stability device vertical flanges 3002, 3006, and 3007, the wing stability device bottom flange 3003, the wing stability device cover 3004, and the wing stability device bumper 3005. In this and other embodiments, the wing stability device may optionally be configured to attach to the base of a disinfection device to help prevent the device from tipping over under applied later loads at the top of the device. Optionally, in this and other embodiments, the wing stability device flange has a relatively narrow profile from top to bottom and extends outwards from the base of the panel to which it is attached at an angle of about 45 degrees. In one example, the first center flange, the second center flange, and the third center flange extend between the bottom flange and the plate at an angle of about 45 degrees. In this and other embodiments, this is sufficient to transfer any lateral forces on the disinfection device or column to the floor and prevent the panel, and the whole assembly, from tipping over. However, this precise angle is not essential for this purpose and other embodiments with different angles, or being perfectly vertical with no angles, would be sufficient for the wing stability device to perform the same function. The top flange may optionally be attached to the base of the panel with screws, or other means of attachment, such as weldments, glue, or bolts and nuts, may be utilized. The length to which the wing stability device extends outwards from the base of the panel assures that the panel can take lateral loads, such as pushing or a person leaning on a panel, without tipping past the point at which the bottom flange of the wing stability device contacts the floor.

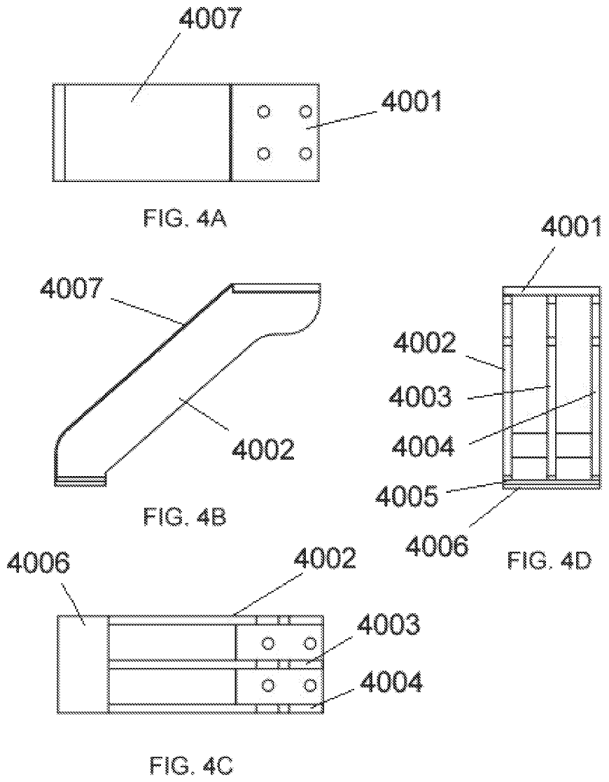

FIGS. 4A through 4D show multiple views of a wing stability device assembly. FIG. 4A illustrates a top view showing the cover plate 4007 and the top flange 4001 containing the screw holes for attaching the wing stability device to the base of the panel. The cover plate in this embodiment is rectangular but this shape is by no means the only possible shape for the flange, nor is the location of the screw holes the only location for the screw holes. Alternate shapes for the top flange are possible, including square, circular, trapezoidal, or irregular. FIG. 4B illustrates a side view of the wing stability device in which the 45 degree angle of the body is evident. In this and other embodiments, the forces transmitted through the top flange, the weight plus any horizontal or vertical forces, may optionally be transmitted through the body of the wing stability device to the floor, and the wing stability device, and may thereby keep the panel, and the entire assembly, from tipping over. Optionally, in this and other embodiments, this stability device may work in conjunction with the column stability device, depending on where the lateral force is applied. FIG. 4C illustrates a bottom view of the wing stability device showing the bottom bumper 4006, and the three center flanges 4002, 4003, and 4004. The location of the center flanges limits the possible location of the four screw holes, but this by no means requires the screws holes to be placed exactly at these locations, nor does it require that screws, or nuts and bolts, be used for this purpose as any means of fixing the top flange to the base of the panel, such as weldments or glue, or magnetic coupling, will serve the purpose equally well. FIG. 4D illustrates a rear or back view of the wing stability device in which the top plate 4001 is shown, the three vertical flanges 4002, 4003, and 4004, the bottom flange 4005 and the bottom bumper 4006. The location and orientation of the center flanges in the current embodiment is primarily a function of the nature of the design, it being composed of seven plates, but alternate shapes and alternate shapes of parts are also possible as long as they provide the same essential function of the current embodiment to resist the tipping of the supported structure, whether that structure be a system, column, a panel, or any other structure of approximately 100 inches tall, 90 inches tall, 80 inches tall, or 79, 78, 77, 76, 75, 74, 73, 72, 71 and 70 inches tall, 60 inches tall, 50 inches tall, 40 inches tall, or 30 inches tall and any height between those listed. That is to say, the wing stability devices, in isolation or in combination with other stability devices such as the column stability devices or column lateral stability devices, provide a means of stabilizing any solid structure or assembly of structures that must resist tipping under lateral or other loads in order to remain functional.

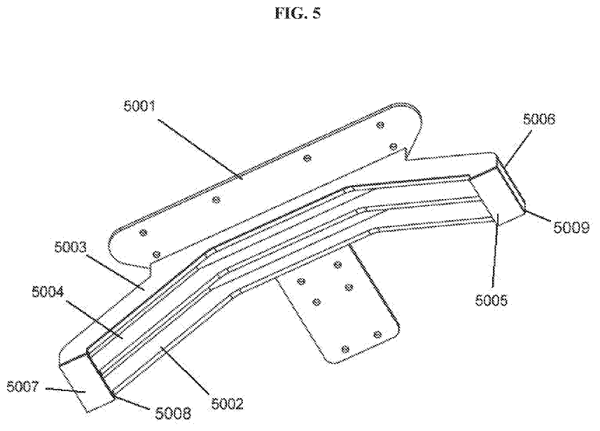

FIG. 5 shows a perspective view of an exemplary embodiment of a column lateral stability device assembly. The column lateral stability device comprises a lateral stability device top flange 5001, the three lateral stability device vertical flanges 5002, 5003, and 5004, the lateral stability device bottom flange 5008, the lateral stability device bottom flange 5009, the lateral stability device bumper 5005, the lateral stability device bumper 5007, and the lateral stability device front cover 5006. In this and other embodiments, the wing stability device may optionally be configured to attach to the base of a disinfection device to help prevent the device from tipping over under applied lateral loads at the top of the device. Optionally, in this and other embodiments, the column lateral stability device will permit the structure to which it is attached to tip a certain amount. In this particular case, the structure may tip about 3-6 degrees from the vertical without tipping over, but the degree to which a structure may be allowed to tip without tipping over is largely dependent on its geometry, weight, and center of gravity, and therefore the actual permissible tipping angle is not a defining characteristic of this invention. The angle at which tipping is designed to stop in this device depends on the geometry of the stability device and in this case the geometry of the stability device. As depicted in FIG. 5, the lateral bottom bumper may optionally contact the floor at an angle of approximately 3-6 degrees, at which point all the lateral and horizontal forces applied to the supported structure may optionally be transmitted through the lateral stability device to the floor. The top flange 5001 is drilled through with holes at various locations for attachment to the base of the supported structure with screws, although the use of screws by no means is the only method of attachment as nuts and bolts, or weldments, or glue, or any other means of attachment that would bear the expected loading would be equally suitable. Nor is the actual location of the screw holes an absolute necessity for the proper functioning of the stability device as any set of hole locations that permit the stability device to perform its intended function will be equally functional,

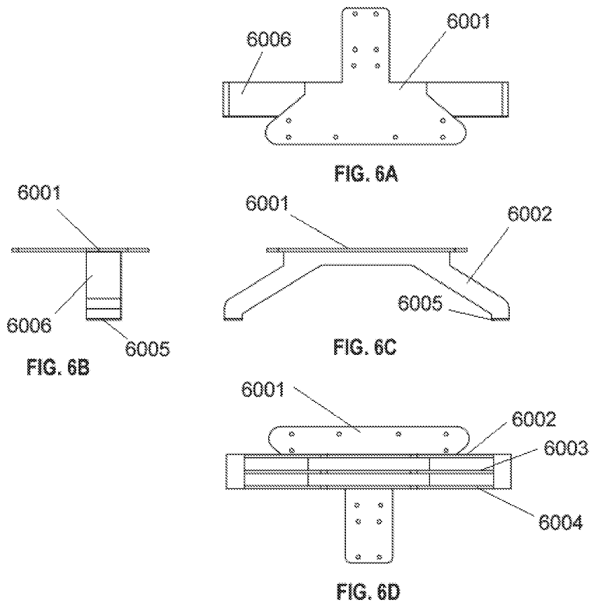

FIGS. 6A through 6D shows multiple perspectives of an exemplary embodiment of a column lateral stability device assembly. FIG. 6A illustrates a top view of the column lateral stability device showing the top flange 6001 and the stability device front cover, which in the instant embodiment performs the function of solidifying the overall assembly of flanges and offers a smooth surface and a low profile to minimize any possible tripping hazards. Optionally, in this and other embodiments, the shape of the column lateral structure is such that it will bear both horizontal and lateral forces applied to the supported structure and transmit them to the floor once it has tipped the permissible tipping distance or angle. None of the individual plates or parts of the column lateral stability device is absolutely essential as long as the structure as a whole performs its intended function, and embodiments that may lack one or more of the designated parts may still function provided the overall strength and integrity of the stability device is maintained. It is possible, for example, for the entire stability device to be manufactured not from nine (9) parts as in this embodiment but from as little as one part. FIG. 6B illustrates a side view of the column lateral stability device that includes the top flange 6001, the stability device cover plate 6006, and the stability device bumper 6005. FIG. 6C illustrates a front view of the column lateral stability device showing the top flange 6001, one of three vertical flanges 6002, and the stability device bumper 6005. The shape evident in FIG. 6C makes it clear that any vertical or horizontal forces placed on the supported structure once it has tipped to the maximum tipping distance or angle will be transmitted to the floor and at that point the column lateral stability device will optionally resist further tipping and will maintain the structure in a stable condition. FIG. 6D illustrates the bottom view of the column lateral stability device showing the top flange 6001 and the center flanges 6002, 6003, and 6004.

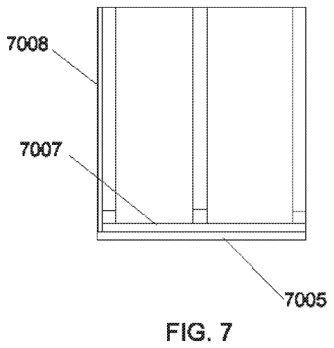

FIG. 7 shows a section of an exemplary embodiment of a column lateral stability device. The stability device comprises a lateral stability device bottom flange 7007 the lateral stability device bumper 7005, and the lateral stability device front cover 7008. This detail illustrates how optionally in this and other embodiments, the bottom flange may fit to the bottom bumper and the cover plate such that no outside edges are unduly exposed and all surfaces fit tightly together.

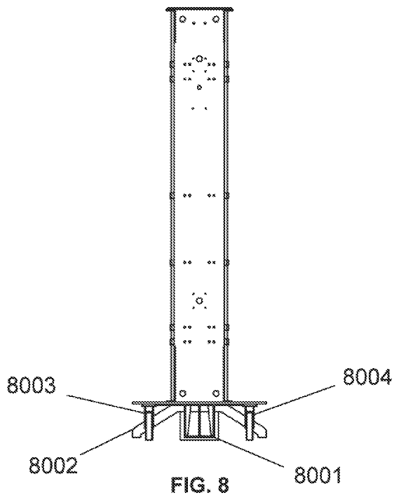

FIG. 8 shows an end view of an exemplary embodiment of a stability device 8001 and a column lateral stability device 8002 on an exemplary embodiment of a disinfection device which includes two components that are not part of the stability device system, the casters 8003 and 8004. Optionally, in this and other embodiments, the stability device will resist tipping in the forward direction while the column lateral stability device will resist tipping in the side direction (that is, once the maximum tipping angle has been reached in either case). Without stability devices in place the column may be subject to tipping over, a hazard to the system or disinfection device, and to surrounding personnel.

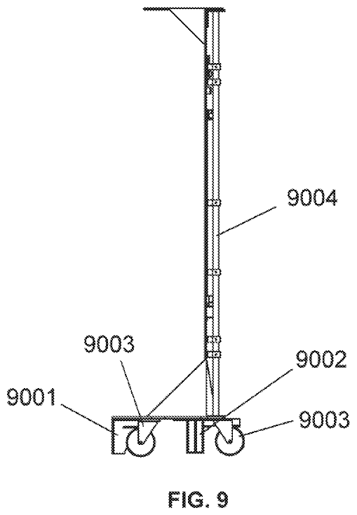

FIG. 9 shows a side view of an exemplary embodiment of a column framework 9004 with exemplary embodiments of both a stability device 9001 and the lateral stability device 9002, shown alongside the casters 9003. The stability device is located at the front of the column framework 9004 and acts to block forward tipping. The column lateral stability device extends to both sides of the base of the column framework 9004 and acts to limit tipping to the sides. Additional stability devices can be added to the assembly throughout to be better suited for the geometry of the systems and counteract the forces from several directions.

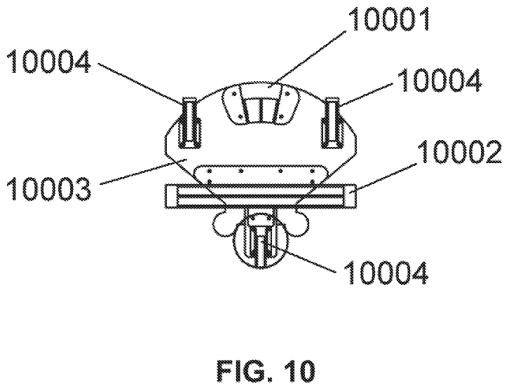

FIG. 10 shows an overhead wireframe view of the column base 10003 to which an exemplary embodiment of a stability device 10001 and a column lateral stability device 10002 are attached. Also indicated in FIG. 10 are three casters 10004. The stability device 10001 is vertically flush with the edge of column base 10003 so as not to create a tripping hazard. The two stability devices 10001 and 10002 are positioned in between the three casters 10004 to prevent tipping in the major possible tipping directions—since the casters form the corners of a hypothetical triangle of vertical support the sides of numerous tipping directions and the two stability devices 10001 and 10002 provide three points of contact with the floor in the event the columns tips too far out there may be more points of contact. There are several possible variations in the number and locations of the stability devices depending on the locations of the casters or other structural supports. By way of example, if the structure had a single normal support point, or two support points, then there may be a minimum of three points where the stability device bottoms must contact the floor to prevent tipping. Optionally, in this or other embodiments, if the structure had four support points (e.g., four wheels or four legs) then four stability device support points may be required. From three support points onwards the minimum number of stability device bottoms may, in this and other embodiments, equal the number of support points. For example, five legs or wheels may require a minimum of five stability device support points, and six legs may require a minimum of six stability device support points, etc. In the case of the current embodiment of the disinfection unit, which has a central column to which are attached two hinged door or panels, additional lateral stability devices are attached to the base of the panels to provide additional stability in the event the whole structure tips.

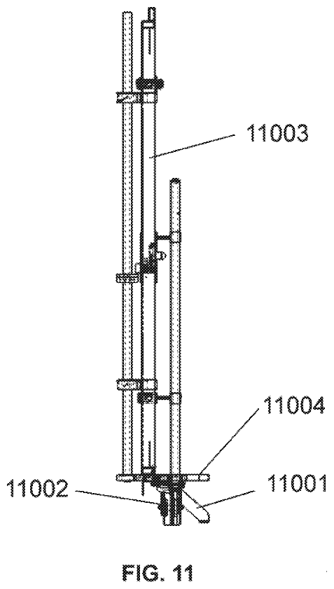

FIG. 11 shows a side view of an exemplary embodiment of a panel 11003 resting on a caster base plate 11004 and a single caster 11002 with an exemplary embodiment of a wing stability device 11003, The wing stability device 11003 prevents tipping on one side, which is the forward side of the central column to which the panel is attached by hinges. The wing stability device 11003 is vertically flush with the edge of the panel caster base plate 11004 so as not to create an undue tripping hazard. Any lateral load placed on the panel (on the left or right side of the image in FIG. 11) will be transferred through the hinges to the central column where the stability devices, in conjunction with the wing stability devices, may act to limit any tipping of the entire assembly. Any lateral load placed on the column will be partly transferred to the panel through the hinges where the wing stability devices will act to help limit tipping of both the panels and the central column.

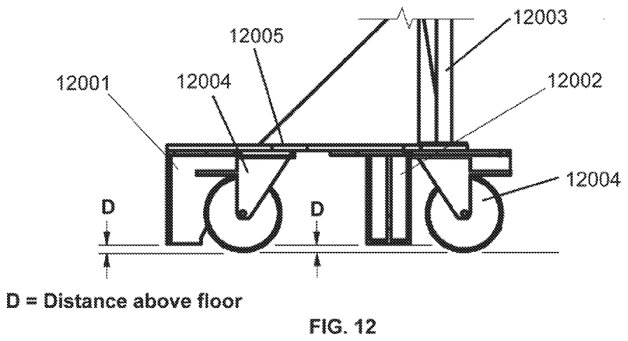

FIG. 12 shows a sectional side view of the base of an exemplary embodiment of a column 12003 with exemplary embodiments of both a stability device 12001 and a lateral stability device 12002 with casters 12004 in place. The height of the stability device 12001 and the lateral stability device 12002 above the floor is indicated by the distance “D” which provides a nominal vertical clearance sufficient to permit easy movement while simultaneously preventing tipping beyond a certain tipping angle. Optionally, in this and other embodiments, the maximum tipping angle to which the structure may be limited is a function of the height, weight, geometry, center of gravity, and expected lateral loading force applied and this will be unique for each application. In the current embodiment of the disinfection unit, the maximum tipping angle (the angle to which the structure is limited by the stability devices) of the center column in the forward direction has been established analytically and empirically to be approximately 3.5 degrees in the forward direction and in both the side or lateral directions (Left and Right sides). The tipping angle for other embodiments may be any angle greater or lesser than 3.5 degrees from the vertical depending on the factors mentioned previously and so the stability device design is not dependent on this particular tipping angle.

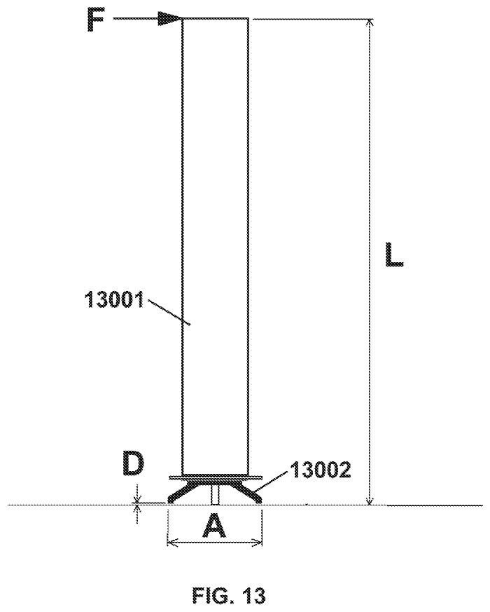

FIG. 13 shows an exemplary schematic side view of a particular embodiment of a column 13001 and the wing stability device 13002 attached to the base of the column as the column is tipped towards the Right, by some lateral load “F” applied at the upper edge of the structure, up to the point the wing stability device bottom bumper on the right contacts the floor. The force F required to tip the structure can be determined from the weight W (in kg), the height H of the column, and the width of the base A (the width of the side casters) if we assume the friction force between the floor and the column keeps the column from sliding. The sum of the moments about the tipping corner on the lower right must equal zero and so the force required to tip the structure initially (before it tips to the maximum tipping angle of 3.5 degrees) is equal to WA/(2H). The weight is 470 lbs. maximum or 235 kg. The column width between the casters is 9.4″ or 0.2388 m. The height of the column is 77.75″ or 1.97 m. Therefore F=(235 kg)(0.2388 m)/(2*1.97 in)=14.24 kg or 31.4 lbs. This is a conservative estimate of the initial tipping force because the center of gravity is actually 30.437″, which is lower than the midpoint of the column, but we will ignore this to be conservative. This computed force, 31.4 lbs., is the force required to tip the column until the wing stability device contacts the floor, at which point the tipping force will increase.

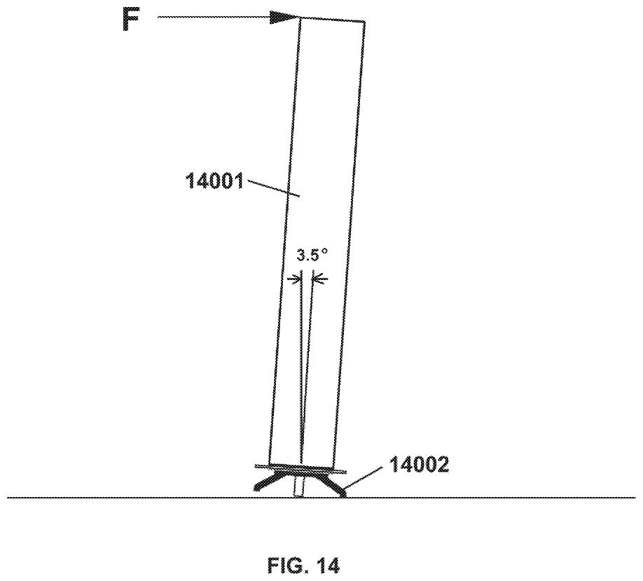

FIG. 14 shows a schematic view of an exemplary embodiment of a column 14001 leaning at the maximum tipping angle of 3.5 degrees at which the wing stability device 14002 will make contact with the floor. This maximum tipping angle has been established by geometry for the lateral wing stability devices, which extend to a width of 19.2″ or 0.488 m. Ignoring the lower center of gravity again and recalculating the tipping force with the new width yields a new tipping force of F=(235 kg)(0.488 m)/(2*1.97 m)=29.1 kg or 64.2 lbs. This estimated force conservatively ignores the effect of the cosine of the 3.5 degree angle on the applied force, but it could be assumed to change with the angle of the column, for conservatism. Clearly, tipping is much inhibited beyond the maximum tipping angle of 3.5 degrees and so the stability device effectively prevents tipping from any reasonable lateral force.

The frame for the disinfection device includes—in some embodiments—one or more of each of the three stability devices: the stability device assembly, the wing stability device assembly, and the column lateral stability device assembly. These assemblies, when attached to the base of the disinfection device, may in some embodiments optionally prevents the device from tipping too far over under applied lateral loads at the top or on the sides of the device. Optionally, in some embodiments, these stability devices may be permanently mounted under the unit or may be releasable, by any one of a number of commonly known mechanical or electrical mechanisms, such that their release facilitates the portability of the unit. In the embodiment in which the stability devices are releasable, their engagement may include, but does not require, direct contact of the stability devices with the floor.

The present embodiment of the stability devices may in some cases offer a passive design for limiting the tipping angle of any structure. It is easily conceivable that in other embodiments that dynamic stability devices could be designed based on these same principles that would employ dampening devices such as levers, springs and pistons, or that may employ solenoids to react dynamically and limit tipping, dampen the tipping motion, or impart an impulse to counter the tipping forces. The linear embodiments of stability devices presented in detail herein and the dynamic variations of stability devices discussed also have analogues in rotational devices or structures in which the rotation of any structure could be limited to a certain rotation angle.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

It is intended that the following claims define the scope of the invention and that methods and structures within the scope of these claims and their equivalents be covered thereby.

1. A stability device connectable to a disinfection device, the stability device comprising
a plate including a plurality of bores, the plate configured to connect the stability device with a base of a disinfection device using the plurality of bores;
a bottom flange substantially parallel with the plate and spaced away therefrom;
a first center flange, a second center flange, and a third center flange each of the first center flange, the second center flange, and the third center flange connected to the plate and the bottom flange; and
a bumper connected to a bottom portion of the bottom flange and configured to contact a Moor when the disinfection device tips.

2. The stability device of claim 1, further comprising:
a stability device cover connected to the plate, the bottom flange, the first center flange, the second center flange, and the third center flange.

3. The stability device of claim 1, wherein the first center flange, the second center flange, and the third center flange extend between the bottom flange and the plate at an angle of about 45 degrees.

4. The stability device of claim 1, wherein first center flange, the second center flange, and the third center flange each have a geometric shape of a trapezoid.

5. A stability device connectable to a disinfection device, the stability device comprising:
a plate configured to connect the stability device with a base of a disinfection device;
a first lateral flange substantially parallel with the plate and spaced away therefrom;
a second lateral flange substantially parallel with the plate and spaced away therefrom; and
a first center flange, a second center flange, and a third center flange each of the first center flange, the second center flange, and the third center flange connected to the plate, the first lateral flange and the second lateral flange.

6. The stability device of claim 5, further comprising:
a first bumper connected to a bottom of the first lateral flange and configured to contact a floor when the disinfection device tips laterally in a first direction.

7. The stability device of claim 5, further comprising:
a second bumper connected to a bottom of the second lateral flange and configured to contact a floor when the disinfection device tips laterally in a second direction.

8. The stability device of claim 5, wherein the first lateral flange is substantially coplanar with the second lateral flange.

9. A stability assembly connectable to a disinfection device, the stability assembly comprising:
a column base including a top portion and a bottom portion opposite the top portion;
a column framework extending substantially vertically from the top portion of the column base;
a stability device connected to the bottom portion of the column base;
a lateral stability device connected to the bottom portion of the column base and spaced away from the stability device.

10. The stability assembly of claim 9, wherein the stability device is positioned at a front portion of the bottom portion of the column base and wherein the lateral stability device is positioned at back portion of the bottom portion of the column base.

11. The stability assembly of claim 9, wherein the lateral stability device further comprises:
a lateral plate configured to connect the lateral stability device to the column base;
a first lateral flange substantially parallel with the lateral plate and spaced away therefrom;
a second lateral flange substantially parallel with the lateral plate and spaced away therefrom; and
a first center flange, a second center flange, and a third center flange each of the first center flange, the second center flange, and the third center flange connected to the plate, the first lateral flange and the second lateral flange.

12. The stability assembly of claim 11, wherein the stability device further comprises:
a stability plate configured to connect the stability device to the column base;
a bottom flange substantially parallel with the stability plate and spaced away therefrom;
a first stability center flange, a second stability center flange, and a third stability center flange each of the stability first center flange, the stability second center flange, and the stability third center flange connected to the stability plate and the bottom flange; and
a bumper connected to a bottom portion of the bottom flange and configured to contact a floor when the stability assembly tips.

13. A method of assembly a stability device connectable to a disinfection device, the method comprising:
providing a plate including a plurality of bores, the plate configured to connect the stability device with a base of a disinfection device using the plurality of bores;
providing a bottom flange configured to be assembled in an arrangement substantially parallel with the plate and configured to be spaced away therefrom;
connecting a first center flange to the plate and the bottom flange; and
connecting a bumper to a bottom portion of the bottom flange, the bumper configured to contact a floor when the disinfection device tips.

14. The method of claim 13, further comprising:
connecting a second center flange to the plate and the bottom flange.

15. The method of claim 14, further comprising:
connecting a third center flange to the plate and the bottom flange.
